(19) United States
(12) Patent Application Publication
(10) Pub. No.: US 2016/0213299 A1
(43) Pub. Date: Jul. 28, 2016

(54) One or More Machines/Articles/Compositions/Processes Related to Traumatic Brain Injuries

(71) Applicant: Elwha LLC, Bellevue, WA (US)

(72) Inventors: Paul G. Allen, Mercer Island, WA (US); Philip V. Bayly, St. Louis, MO (US); David Lozoff Brody, St. Louis, MO (US); Jesse R. Cheatham, III, Seattle, WA (US); Richard Glen Ellenbogen, Seattle, WA (US); Roderick A. Hyde, Redmond, WA (US); Muriel Y. Ishikawa, Livermore, CA (US); Eric C. Leuthardt, St. Louis, MO (US); Nathan P. Myhrvold, Medina, WA (US); Robert C. Petroski, Seattle, WA (US); Raul Radovitzky, Bedford, MA (US); Elizabeth A. Sweeney, Seattle, WA (US); Clarence T. Tegreene, Mercer Island, WA (US); Nicholas W. Touran, Seattle, WA (US); Lowell L. Wood, JR., Bellevue, WA (US); Victoria Y. H. Wood, Livermore, CA (US); Richard T. Lord, Gig Harbor, WA (US); Robert W. Lord, Seattle, WA (US)

(21) Appl. No.: 14/929,338

(22) Filed: Oct. 31, 2015

Related U.S. Application Data
(60) Provisional application No. 62/108,047, filed on Jan. 26, 2015.

(51) Int. Cl. A61B 5/00; A61B 5/11

(52) U.S. Cl. CPC A61B 5/4064; A61B 5/1112; A61B 5/6803; A61B 2503/10

(57) ABSTRACT

A method substantially as shown and described in the detailed description and/or drawings and/or elsewhere herein. A device substantially as shown and described in the detailed description and/or drawings and/or elsewhere herein.

### CROSS-REFERENCE TO RELATED APPLICATIONS

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

For purposes of the USPTO extra-statutory requirements, the present application constitutes a non-provisional of U.S. Patent Application No. 62/108,047, entitled ONE OR MORE MACHINES/ARTICLES/COMPOSITIONS/PROCESSES RELATED TO TRAUMATIC BRAIN INJURIES, naming Paul G. Allen, Philip V. Bayly, David L. Brody, Jesse R. Cheatham, III, Richard G. Ellenbogen, Roderick A. Hyde, Muriel Y. Ishikawa, Eric C. Leuthardt, Richard T. Lord, Robert W. Lord, Nathan P. Myhrvold, Robert C. Petroski, Raul Radovitzky, Elizabeth A. Sweeney, Clarence T. Tegreene, Nicholas W. Touran, Lowell L. Wood, Jr., and Victoria Y. H. Wood, filed 26 Jan. 2015 with attorney docket no. 0414-002-014-PR0001, which is currently co-pending or is an application of which a currently co-pending application is entitled to the benefit of the filing date.

### RELATED APPLICATIONS

None.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### BACKGROUND

This application is related to one or more machines, articles, compositions, and processes related to traumatic brain injuries such as regarding sensing, testing, status, location, and access of players of sports during their games.

### SUMMARY

In one or more various aspects, a method includes but is not limited to that which is illustrated in the drawings. In addition to the foregoing, other method aspects are described in the claims, drawings, and text forming a part of the disclosure set forth herein.

In one or more various aspects, one or more related systems may be implemented in machines, compositions of matter, or manufactures of systems, limited to patentable subject matter under 35 U.S.C. 101. The one or more related systems may include, but are not limited to, circuitry and/or programming for carrying out the herein-referenced method aspects. The circuitry and/or programming may be virtually any combination of hardware, software, and/or firmware configured to effect the herein-referenced method aspects depending upon the design choices of the system designer, and limited to patentable subject matter under 35 USC 101.

In one or more various aspects, a system includes but is not limited to that which is illustrated in the drawings. In addition to the foregoing, other system aspects are described in the claims, drawings, and text forming a part of the disclosure set forth herein.

In one or more various aspects, a computer program product, comprising a signal bearing medium, bearing one or more instructions includes but is not limited to that which is illustrated in the drawings. In addition to the foregoing, other computer program product aspects are described in the claims, drawings, and text forming a part of the disclosure set forth herein.

In one or more various aspects, a device is defined by a computational language, such that the device comprises but is not limited to that which is illustrated in the drawings. In addition to the foregoing, other device aspects are described in the claims, drawings, and text forming a part of the disclosure set forth herein.

In addition to the foregoing, various other method and/or system and/or program product aspects are set forth and described in the teachings such as text (e.g., claims and/or detailed description) and/or drawings of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent by reference to the detailed description, the corresponding drawings, and/or in the teachings set forth herein.

### BRIEF DESCRIPTION OF THE FIGURES

For a more complete understanding of embodiments, reference now is made to the following descriptions taken in connection with the accompanying drawings. The use of the same symbols in different drawings typically indicates similar or identical items, unless context dictates otherwise. The illustrative embodiments described in the detailed description, drawings, and claims are not meant to be limiting. Other embodiments may be utilized, and other changes may be made, without departing from the spirit or scope of the subject matter presented here.

FIG. 1 shows a high-level system diagram of one or more exemplary environments in which transactions and potential transactions may be carried out, according to one or more embodiments. FIG. 1 forms a partially schematic diagram of an environment(s) and/or an implementation(s) of technologies described herein when FIGS. 1-A through 1-F are stitched together in the manner shown in FIG. 1, which is reproduced below in table format.

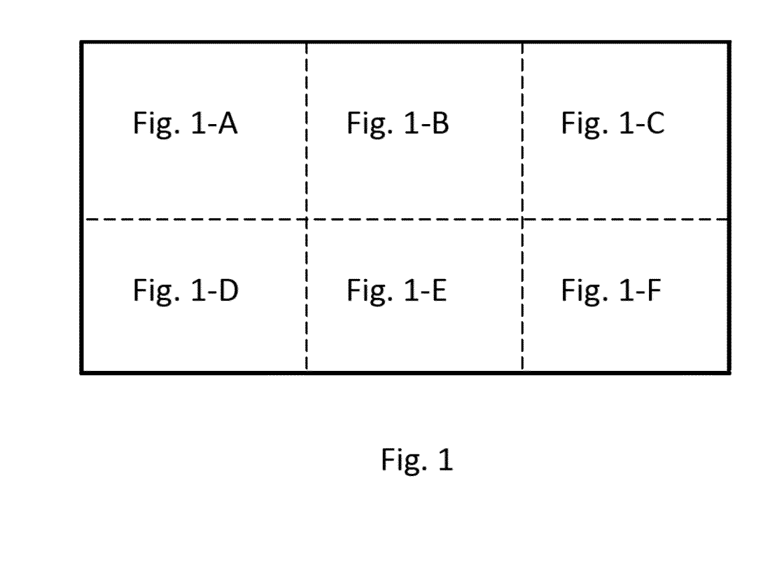

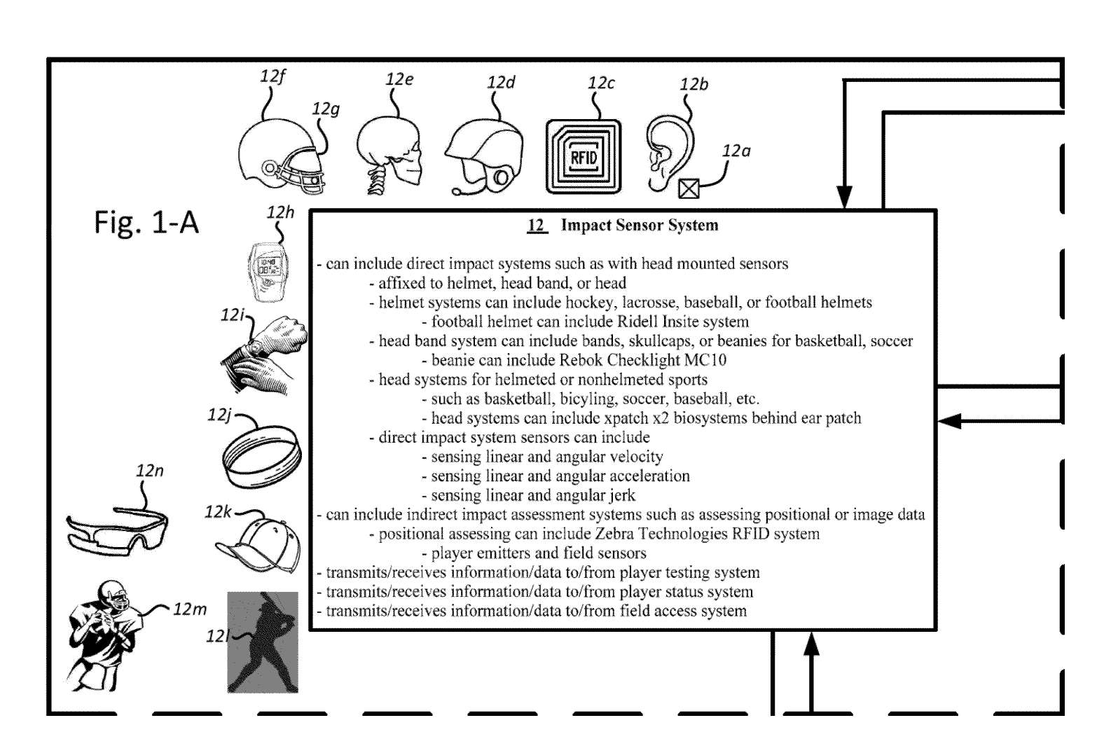

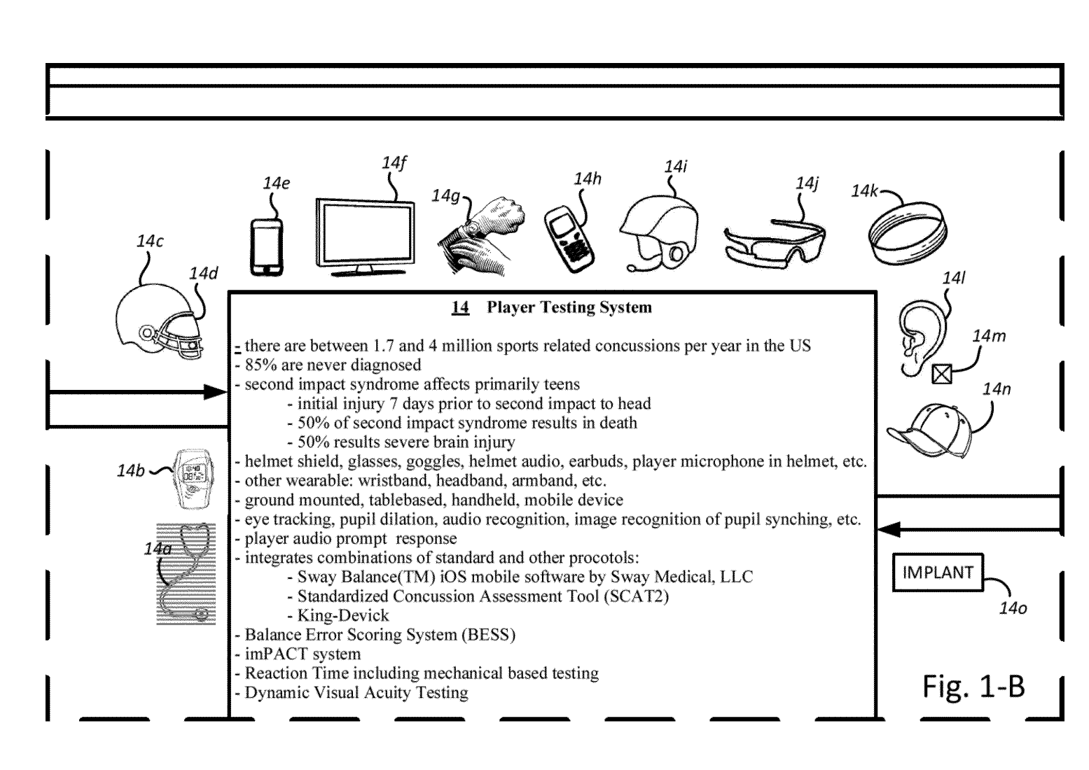

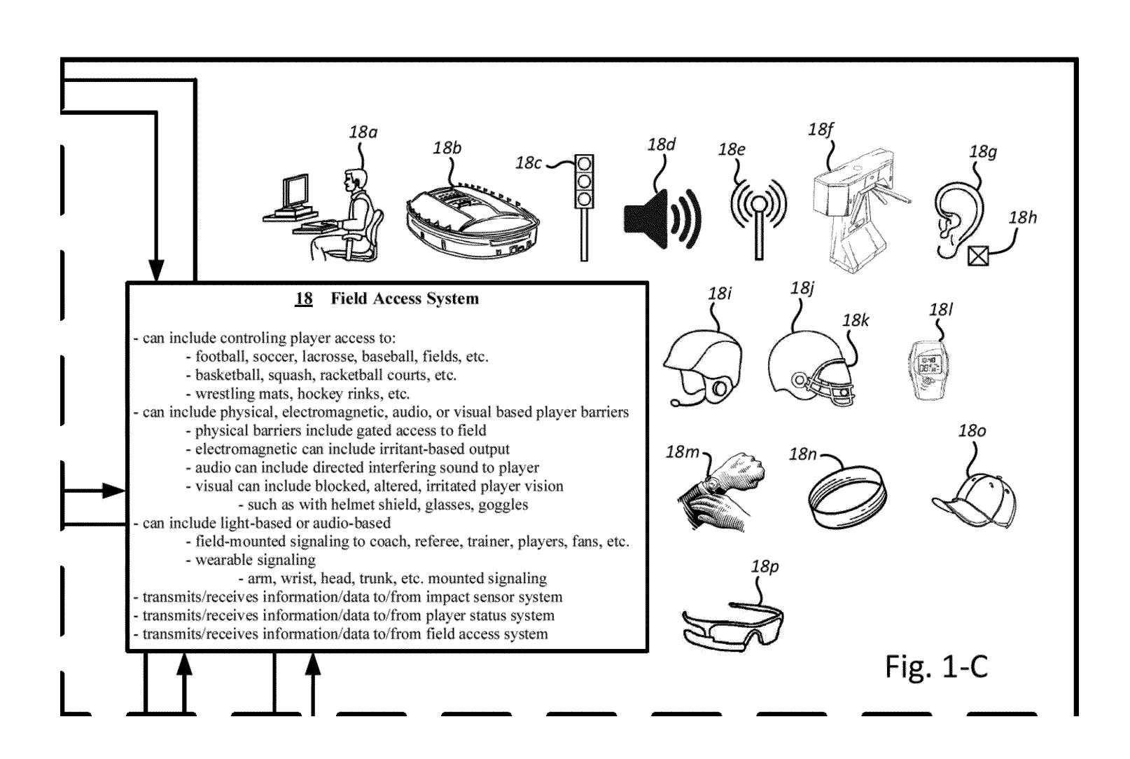

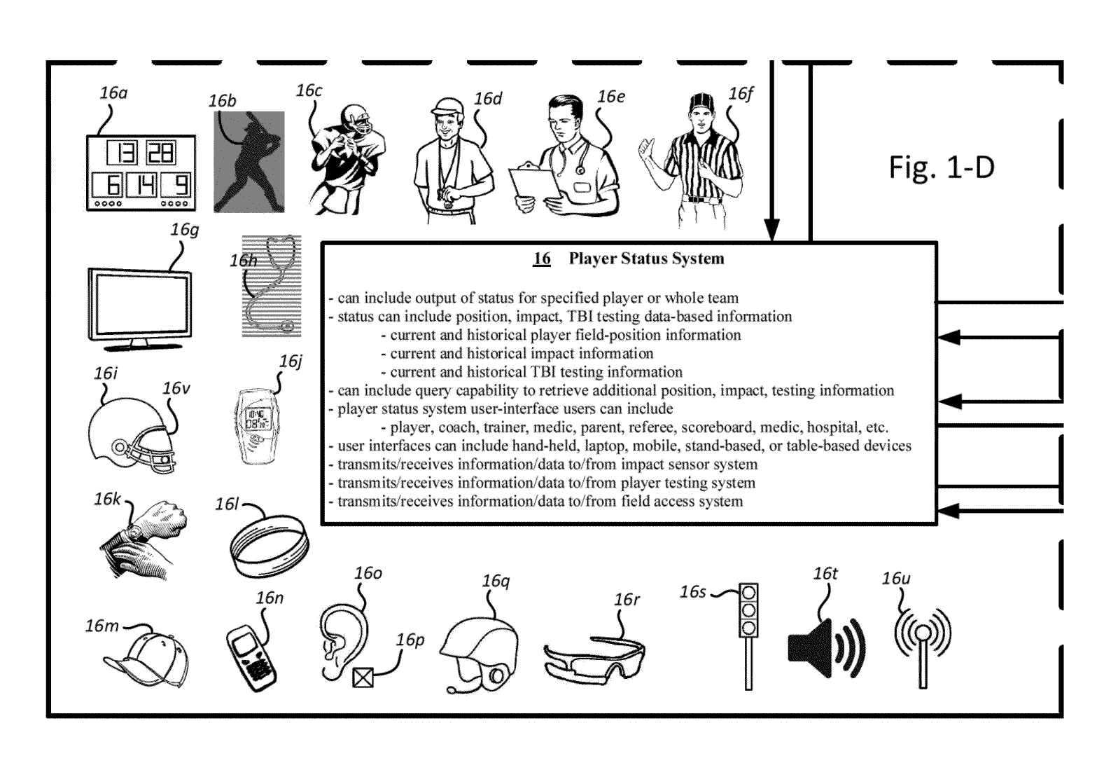

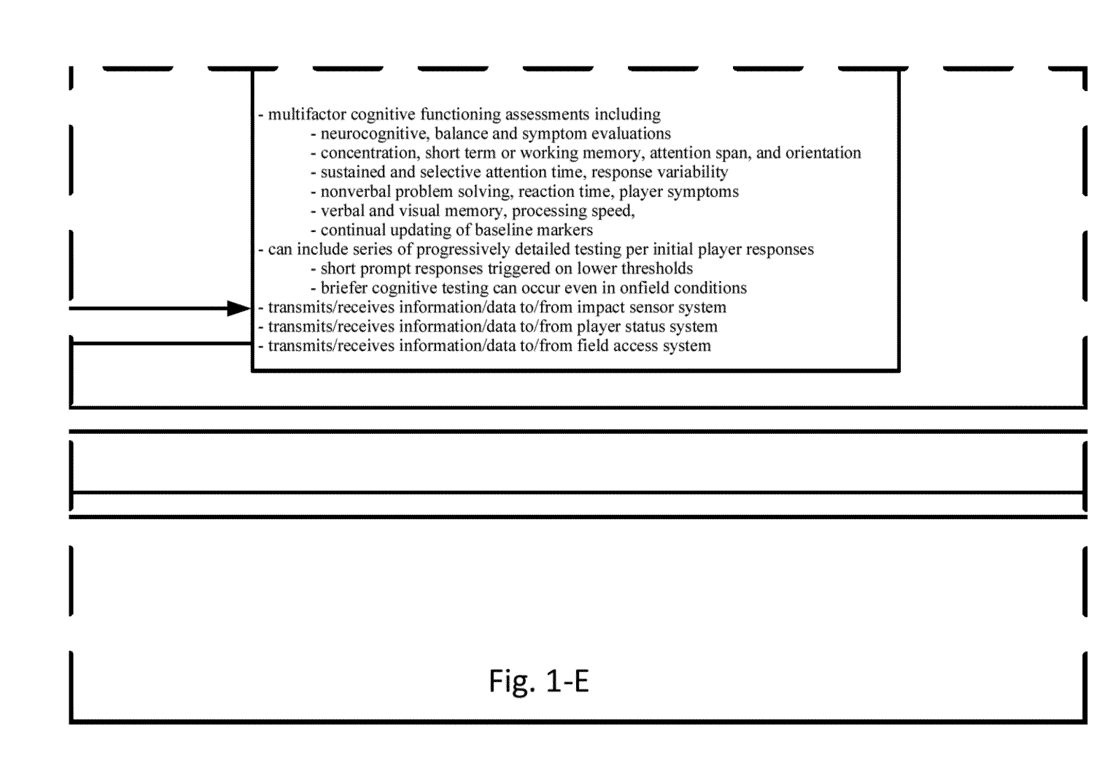

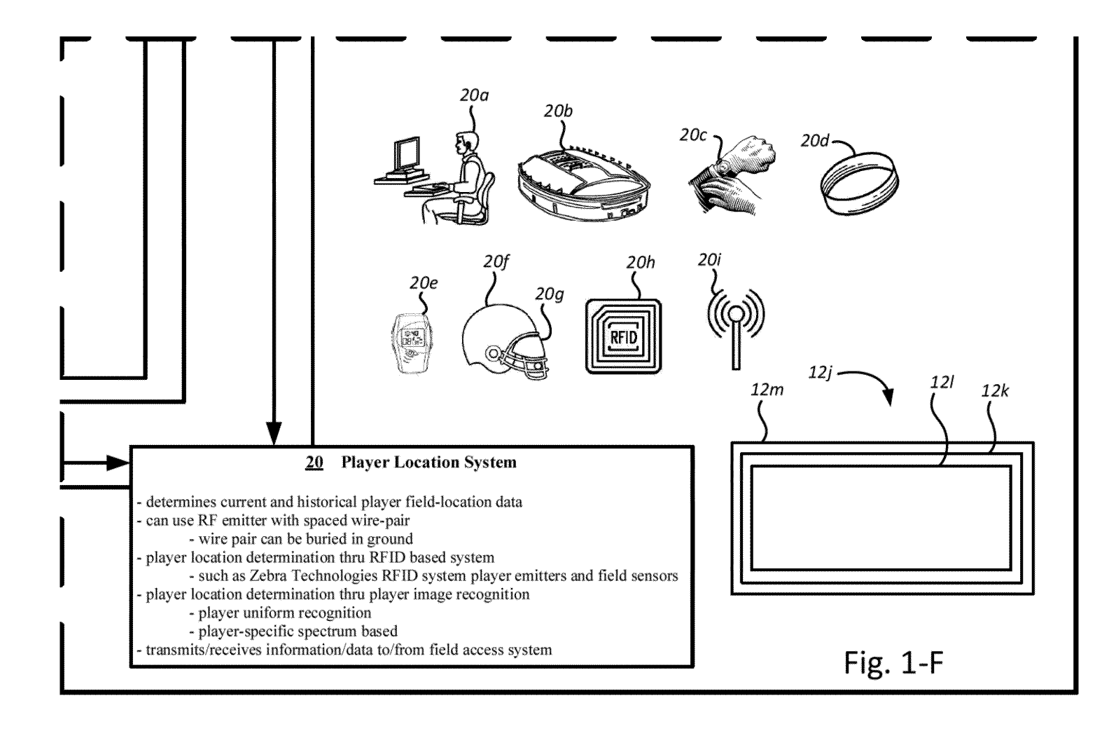

In accordance with 37 C.F.R. §1.84(h)(2), FIG. 1 shows “a view of a large machine or device in its entirety . . . broken into partial views . . . extended over several sheets” labeled FIG. 1-A through FIG. 1-F (Sheets 1-7 including FIG. 1). The “views on two or more sheets form, in effect, a single complete view, [and] the views on the several sheets . . . [are] so arranged that the complete figure can be assembled” from “partial views drawn on separate sheets . . . linked edge to edge. Thus, in FIG. 1, the partial view FIGS. 1-A through 1-F are ordered alphabetically, by increasing in columns from left to right, and increasing in rows top to bottom, as shown in the following table:

TABLE 1
Table showing alignment of enclosed drawings to form partial schematic
of one or more environments.

| Pos. (0,0) | X-Position 1 | X-Position 2 | X-Position 3 |
|---|---|---|---|
| Y-Pos. 1 | (1,1): FIG. | (1,2): FIG. | (1,3): FIG. |
| | 1-A | 1-B | 1-C |
| Y-Pos. 2 | (2,1): FIG. | (2,2): FIG. | (2,3): FIG. |
| | 1-D | 1-E | 1-F |

In accordance with 37 C.F.R. §1.84(h)(2), FIG. 1 is “ . . . a view of a large machine or device in its entirety . . . broken into partial views . . . extended over several sheets . . . [with] no loss in facility of understanding the view.” The partial views drawn on the several sheets indicated in the above table are capable of being linked edge to edge, so that no partial view contains parts of another partial view. As here, “where views on two or more sheets form, in effect, a single complete view, the views on the several sheets are so arranged that the complete figure can be assembled without concealing any part of any of the views appearing on the various sheets.” 37 C.F.R. §1.84(h)(2).

It is noted that one or more of the partial views of the drawings may be blank, or may be absent of substantive elements (e.g., may show only lines, connectors, arrows, and/or the like). These drawings are included in order to assist readers of the application in assembling the single complete view from the partial sheet format required for submission by the USPTO, and, while their inclusion is not required and may be omitted in this or other applications without subtracting from the disclosed matter as a whole, their inclusion is proper, and should be considered and treated as intentional.

FIG. 1-A, when placed at position (1,1), forms at least a portion of a partially schematic diagram of an environment(s) and/or an implementation(s) of technologies described herein.

FIG. 1-B, when placed at position (1,2), forms at least a portion of a partially schematic diagram of an environment(s) and/or an implementation(s) of technologies described herein.

FIG. 1-C, when placed at position (1,3), forms at least a portion of a partially schematic diagram of an environment(s) and/or an implementation(s) of technologies described herein.

FIG. 1-D, when placed at position (2,1), forms at least a portion of a partially schematic diagram of an environment(s) and/or an implementation(s) of technologies described herein.

FIG. 1-E, when placed at position (2,2), forms at least a portion of a partially schematic diagram of an environment(s) and/or an implementation(s) of technologies described herein.

FIG. 1-F, when placed at position (2,3), forms at least a portion of a partially schematic diagram of an environment(s) and/or an implementation(s) of technologies described herein.

### DETAILED DESCRIPTION

### Overview

In the following detailed description, reference is made to the accompanying drawings, which form a part hereof. In the drawings, similar symbols typically identify similar or identical components or items, unless context dictates otherwise. The illustrative embodiments described in the detailed description, drawings, and claims are not meant to be limiting. Other embodiments may be utilized, and other changes may be made, without departing from the spirit or scope of the subject matter presented here.

Thus, in accordance with various embodiments, computationally implemented methods, systems, circuitry, articles of manufacture, ordered chains of matter, and computer program products are designed to, among other things, provide an interface for that substantially as shown and described in the detailed description and/or drawings and/or elsewhere herein.

The claims, description, and drawings of this application may describe one or more of the instant technologies in operational/functional language, for example as a set of operations to be performed by a computer. Such operational/functional description in most instances would be understood by one skilled the art as specifically-configured hardware (e.g., because a general purpose computer in effect becomes a special purpose computer once it is programmed to perform particular functions pursuant to instructions from program software (e.g., a high-level computer program serving as a hardware specification)).

The claims, description, and drawings of this application may describe one or more of the instant technologies in operational/functional language, for example as a set of operations to be performed by a computer. Such operational/functional description in most instances would be understood by one skilled the art as specifically-configured hardware (e.g., because a general purpose computer in effect becomes a special purpose computer once it is programmed to perform particular functions pursuant to instructions from program software).

### Operational/Functional Language is a Concrete Specification for Physical Implementation

Importantly, although the operational/functional descriptions described herein are understandable by the human mind, they are not abstract ideas of the operations/functions divorced from computational implementation of those operations/functions. Rather, the operations/functions represent a specification for the massively complex computational machines or other means. As discussed in detail below, the operational/functional language must be read in its proper technological context, i.e., as concrete specifications for physical implementations.

The logical operations/functions described herein are a distillation of machine specifications or other physical mechanisms specified by the operations/functions such that the otherwise inscrutable machine specifications may be comprehensible to the human mind. The distillation also allows one of skill in the art to adapt the operational/functional description of the technology across many different specific vendors' hardware configurations or platforms, without being limited to specific vendors' hardware configurations or platforms.

Some of the present technical description (e.g., detailed description, drawings, claims, etc.) may be set forth in terms of logical operations/functions. As described in more detail in the following paragraphs, these logical operations/functions are not representations of abstract ideas, but rather representative of static or sequenced specifications of various hardware elements. Differently stated, unless context dictates otherwise, the logical operations/functions will be understood by those of skill in the art to be representative of static or sequenced specifications of various hardware elements. This is true because tools available to one of skill in the art to implement technical disclosures set forth in operational/functional formats—tools in the form of a high-level programming language (e.g., C, java, visual basic), etc.), or tools in the form of Very high speed Hardware Description Language (“VHDL,” which is a language that uses text to describe logic circuits)—are generators of static or sequenced specifications of various hardware configurations. This fact is sometimes obscured by the broad term “software,” but, as shown by the following explanation, those skilled in the art understand that what is termed “software” is a shorthand for a massively complex interchaining/specification of ordered-matter elements. The term “ordered-matter elements” may refer to physical components of computation, such as assemblies of electronic logic gates, molecular computing logic constituents, quantum computing mechanisms, etc.

For example, a high-level programming language is a programming language with strong abstraction, e.g., multiple levels of abstraction, from the details of the sequential organizations, states, inputs, outputs, etc., of the machines that a high-level programming language actually specifies. In order to facilitate human comprehension, in many instances, high-level programming languages resemble or even share symbols with natural languages.

It has been argued that because high-level programming languages use strong abstraction (e.g., that they may resemble or share symbols with natural languages), they are therefore a “purely mental construct.” (e.g., that “software”—a computer program or computer programming—is somehow an ineffable mental construct, because at a high level of abstraction, it can be conceived and understood in the human mind). This argument has been used to characterize technical description in the form of functions/operations as somehow “abstract ideas.” In fact, in technological arts (e.g., the information and communication technologies) this is not true.

The fact that high-level programming languages use strong abstraction to facilitate human understanding should not be taken as an indication that what is expressed is an abstract idea. In fact, those skilled in the art understand that just the opposite is true. If a high-level programming language is the tool used to implement a technical disclosure in the form of functions/operations, those skilled in the art will recognize that, far from being abstract, imprecise, “fuzzy,” or “mental” in any significant semantic sense, such a tool is instead a near incomprehensibly precise sequential specification of specific computational machines—the parts of which are built up by activating/selecting such parts from typically more general computational machines over time (e.g., clocked time). This fact is sometimes obscured by the superficial similarities between high-level programming languages and natural languages. These superficial similarities also may cause a glossing over of the fact that high-level programming language implementations ultimately perform valuable work by creating/controlling many different computational machines.

The many different computational machines that a high-level programming language specifies are almost unimaginably complex. At base, the hardware used in the computational machines typically consists of some type of ordered matter (e.g., traditional electronic devices (e.g., transistors), deoxyribonucleic acid (DNA), quantum devices, mechanical switches, optics, fluidics, pneumatics, optical devices (e.g., optical interference devices), molecules, etc.) that are arranged to form logic gates. Logic gates are typically physical devices that may be electrically, mechanically, chemically, or otherwise driven to change physical state in order to create a physical reality of Boolean logic.

Logic gates may be arranged to form logic circuits, which are typically physical devices that may be electrically, mechanically, chemically, or otherwise driven to create a physical reality of certain logical functions. Types of logic circuits include such devices as multiplexers, registers, arithmetic logic units (ALUs), computer memory, etc., each type of which may be combined to form yet other types of physical devices, such as a central processing unit (CPU)—the best known of which is the microprocessor. A modern microprocessor will often contain more than one hundred million logic gates in its many logic circuits (and often more than a billion transistors).

The logic circuits forming the microprocessor are arranged to provide a microarchitecture that will carry out the instructions defined by that microprocessor's defined Instruction Set Architecture. The Instruction Set Architecture is the part of the microprocessor architecture related to programming, including the native data types, instructions, registers, addressing modes, memory architecture, interrupt and exception handling, and external Input/Output.

The Instruction Set Architecture includes a specification of the machine language that can be used by programmers to use/control the microprocessor. Since the machine language instructions are such that they may be executed directly by the microprocessor, typically they consist of strings of binary digits, or bits. For example, a typical machine language instruction might be many bits long (e.g., 32, 64, or 128 bit strings are currently common). A typical machine language instruction might take the form “11110000101011110000111100111111” (a 32 bit instruction).

It is significant here that, although the machine language instructions are written as sequences of binary digits, in actuality those binary digits specify physical reality. For example, if certain semiconductors are used to make the operations of Boolean logic a physical reality, the apparently mathematical bits “1” and “0” in a machine language instruction actually constitute shorthand that specifies the application of specific voltages to specific wires. For example, in some semiconductor technologies, the binary number “1” (e.g., logical “1”) in a machine language instruction specifies around +5 volts applied to a specific “wire” (e.g., metallic traces on a printed circuit board) and the binary number “0” (e.g., logical “0”) in a machine language instruction specifies around −5 volts applied to a specific “wire.” In addition to specifying voltages of the machines' configuration, such machine language instructions also select out and activate specific groupings of logic gates from the millions of logic gates of the more general machine. Thus, far from abstract mathematical expressions, machine language instruction programs, even though written as a string of zeros and ones, specify many, many constructed physical machines or physical machine states.

Machine language is typically incomprehensible by most humans (e.g., the above example was just ONE instruction, and some personal computers execute more than two billion instructions every second). Thus, programs written in machine language—which may be tens of millions of machine language instructions long—are incomprehensible. In view of this, early assembly languages were developed that used mnemonic codes to refer to machine language instructions, rather than using the machine language instructions' numeric values directly (e.g., for performing a multiplication operation, programmers coded the abbreviation “mult,” which represents the binary number “011000” in MIPS machine code). While assembly languages were initially a great aid to humans controlling the microprocessors to perform work, in time the complexity of the work that needed to be done by the humans outstripped the ability of humans to control the microprocessors using merely assembly languages.

At this point, it was noted that the same tasks needed to be done over and over, and the machine language necessary to do those repetitive tasks was the same. In view of this, compilers were created. A compiler is a device that takes a statement that is more comprehensible to a human than either machine or assembly language, such as “add 2+2 and output the result,” and translates that human understandable statement into a complicated, tedious, and immense machine language code (e.g., millions of 32, 64, or 128 bit length strings). Compilers thus translate high-level programming language into machine language.

This compiled machine language, as described above, is then used as the technical specification which sequentially constructs and causes the interoperation of many different computational machines such that humanly useful, tangible, and concrete work is done. For example, as indicated above, such machine language—the compiled version of the higher-level language—functions as a technical specification which selects out hardware logic gates, specifies voltage levels, voltage transition timings, etc., such that the humanly useful work is accomplished by the hardware.

Thus, a functional/operational technical description, when viewed by one of skill in the art, is far from an abstract idea. Rather, such a functional/operational technical description, when understood through the tools available in the art such as those just described, is instead understood to be a humanly understandable representation of a hardware specification, the complexity and specificity of which far exceeds the comprehension of most any one human. With this in mind, those skilled in the art will understand that any such operational/functional technical descriptions—in view of the disclosures herein and the knowledge of those skilled in the art—may be understood as operations made into physical reality by (a) one or more interchained physical machines, (b) interchained logic gates configured to create one or more physical machine(s) representative of sequential/combinatorial logic(s), (c) interchained ordered matter making up logic gates (e.g., interchained electronic devices (e.g., transistors), DNA, quantum devices, mechanical switches, optics, fluidics, pneumatics, molecules, etc.) that create physical reality representative of logic(s), or (d) virtually any combination of the foregoing. Indeed, any physical object which has a stable, measurable, and changeable state may be used to construct a machine based on the above technical description. Charles Babbage, for example, constructed the first computer out of wood and powered by cranking a handle.

Thus, far from being understood as an abstract idea, those skilled in the art will recognize a functional/operational technical description as a humanly-understandable representation of one or more almost unimaginably complex and time sequenced hardware instantiations. The fact that functional/operational technical descriptions might lend themselves readily to high-level computing languages (or high-level block diagrams for that matter) that share some words, structures, phrases, etc. with natural language simply cannot be taken as an indication that such functional/operational technical descriptions are abstract ideas, or mere expressions of abstract ideas. In fact, as outlined herein, in the technological arts this is simply not true. When viewed through the tools available to those of skill in the art, such functional/operational technical descriptions are seen as specifying hardware configurations of almost unimaginable complexity.

As outlined above, the reason for the use of functional/operational technical descriptions is at least twofold. First, the use of functional/operational technical descriptions allows near-infinitely complex machines and machine operations arising from interchained hardware elements to be described in a manner that the human mind can process (e.g., by mimicking natural language and logical narrative flow). Second, the use of functional/operational technical descriptions assists the person of skill in the art in understanding the described subject matter by providing a description that is more or less independent of any specific vendor's piece(s) of hardware.

The use of functional/operational technical descriptions assists the person of skill in the art in understanding the described subject matter since, as is evident from the above discussion, one could easily, although not quickly, transcribe the technical descriptions set forth in this document as trillions of ones and zeroes, billions of single lines of assembly-level machine code, millions of logic gates, thousands of gate arrays, or any number of intermediate levels of abstractions. However, if any such low-level technical descriptions were to replace the present technical description, a person of skill in the art could encounter undue difficulty in implementing the disclosure, because such a low-level technical description would likely add complexity without a corresponding benefit (e.g., by describing the subject matter utilizing the conventions of one or more vendor-specific pieces of hardware). Thus, the use of functional/operational technical descriptions assists those of skill in the art by separating the technical descriptions from the conventions of any vendor-specific piece of hardware.

In view of the foregoing, the logical operations/functions set forth in the present technical description are representative of static or sequenced specifications of various ordered-matter elements, in order that such specifications may be comprehensible to the human mind and adaptable to create many various hardware configurations. The logical operations/functions disclosed herein should be treated as such, and should not be disparagingly characterized as abstract ideas merely because the specifications they represent are presented in a manner that one of skill in the art can readily understand and apply in a manner independent of a specific vendor's hardware implementation.

Those having skill in the art will recognize that the state of the art has progressed to the point where there is little distinction left between hardware, software (e.g., a high-level computer program serving as a hardware specification), and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software (e.g., a high-level computer program serving as a hardware specification), and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software (e.g., a high-level computer program serving as a hardware specification) implementation; or, yet again alternatively, the implementer may opt for some combination of hardware, software (e.g., a high-level computer program serving as a hardware specification), and/or firmware in one or more machines, compositions of matter, and articles of manufacture, limited to patentable subject matter under 35 USC 101. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software (e.g., a high-level computer program serving as a hardware specification), and or firmware.

In some implementations described herein, logic and similar implementations may include computer programs or other control structures. Electronic circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software (e.g., a high-level computer program serving as a hardware specification) or firmware, or of gate arrays or programmable hardware, such as by performing a reception of or a transmission of one or more instructions in relation to one or more operations described herein. Alternatively or additionally, in some variants, an implementation may include special-purpose hardware, software (e.g., a high-level computer program serving as a hardware specification), firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or invoking circuitry for enabling, triggering, coordinating, requesting, or otherwise causing one or more occurrences of virtually any functional operation described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled/implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

The term module, as used in the foregoing/following disclosure, may refer to a collection of one or more components that are arranged in a particular manner, or a collection of one or more general-purpose components that may be configured to operate in a particular manner at one or more particular points in time, and/or also configured to operate in one or more further manners at one or more further times. For example, the same hardware, or same portions of hardware, may be configured/reconfigured in sequential/parallel time(s) as a first type of module (e.g., at a first time), as a second type of module (e.g., at a second time, which may in some instances coincide with, overlap, or follow a first time), and/or as a third type of module (e.g., at a third time which may, in some instances, coincide with, overlap, or follow a first time and/or a second time), etc. Reconfigurable and/or controllable components (e.g., general purpose processors, digital signal processors, field programmable gate arrays, etc.) are capable of being configured as a first module that has a first purpose, then a second module that has a second purpose and then, a third module that has a third purpose, and so on. The transition of a reconfigurable and/or controllable component may occur in as little as a few nanoseconds, or may occur over a period of minutes, hours, or days.

In some such examples, at the time the component is configured to carry out the second purpose, the component may no longer be capable of carrying out that first purpose until it is reconfigured. A component may switch between configurations as different modules in as little as a few nanoseconds. A component may reconfigure on-the-fly, e.g., the reconfiguration of a component from a first module into a second module may occur just as the second module is needed. A component may reconfigure in stages, e.g., portions of a first module that are no longer needed may reconfigure into the second module even before the first module has finished its operation. Such reconfigurations may occur automatically, or may occur through prompting by an external source, whether that source is another component, an instruction, a signal, a condition, an external stimulus, or similar.

For example, a central processing unit of a personal computer may, at various times, operate as a module for displaying graphics on a screen, a module for writing data to a storage medium, a module for receiving user input, and a module for multiplying two large prime numbers, by configuring its logical gates in accordance with its instructions. Such reconfiguration may be invisible to the naked eye, and in some embodiments may include activation, deactivation, and/or re-routing of various portions of the component, e.g., switches, logic gates, inputs, and/or outputs. Thus, in the examples found in the foregoing/following disclosure, if an example includes or recites multiple modules, the example includes the possibility that the same hardware may implement more than one of the recited modules, either contemporaneously or at discrete times or timings. The implementation of multiple modules, whether using more components, fewer components, or the same number of components as the number of modules, is merely an implementation choice and does not generally affect the operation of the modules themselves. Accordingly, it should be understood that any recitation of multiple discrete modules in this disclosure includes implementations of those modules as any number of underlying components, including, but not limited to, a single component that reconfigures itself over time to carry out the functions of multiple modules, and/or multiple components that similarly reconfigure, and/or special purpose reconfigurable components.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof, limited to patentable subject matter under 35 U.S.C. 101; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs (e.g., graphene based circuitry). Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will recognize that at least a portion of the devices and/or processes described herein can be integrated into an image processing system. Those having skill in the art will recognize that a typical image processing system generally includes one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art will recognize that at least a portion of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

Those skilled in the art will recognize that at least a portion of the devices and/or processes described herein can be integrated into a mote system. Those having skill in the art will recognize that a typical mote system generally includes one or more memories such as volatile or non-volatile memories, processors such as microprocessors or digital signal processors, computational entities such as operating systems, user interfaces, drivers, sensors, actuators, applications programs, one or more interaction devices (e.g., an antenna USB ports, acoustic ports, etc.), control systems including feedback loops and control motors (e.g., feedback for sensing or estimating position and/or velocity; control motors for moving and/or adjusting components and/or quantities). A mote system may be implemented utilizing suitable components, such as those found in mote computing/communication systems. Specific examples of such components entail such as Intel Corporation's and/or Crossbow Corporation's mote components and supporting hardware, software, and/or firmware.

For the purposes of this application, “cloud” computing may be understood as described in the cloud computing literature. For example, cloud computing may be methods and/or systems for the delivery of computational capacity and/or storage capacity as a service. The “cloud” may refer to one or more hardware and/or software components that deliver or assist in the delivery of computational and/or storage capacity, including, but not limited to, one or more of a client, an application, a platform, an infrastructure, and/or a server The cloud may refer to any of the hardware and/or software associated with a client, an application, a platform, an infrastructure, and/or a server. For example, cloud and cloud computing may refer to one or more of a computer, a processor, a storage medium, a router, a switch, a modem, a virtual machine (e.g., a virtual server), a data center, an operating system, a middleware, a firmware, a hardware back-end, a software back-end, and/or a software application. A cloud may refer to a private cloud, a public cloud, a hybrid cloud, and/or a community cloud. A cloud may be a shared pool of configurable computing resources, which may be public, private, semi-private, distributable, scaleable, flexible, temporary, virtual, and/or physical. A cloud or cloud service may be delivered over one or more types of network, e.g., a mobile communication network, and the Internet.

As used in this application, a cloud or a cloud service may include one or more of infrastructure-as-a-service (“IaaS”), platform-as-a-service (“PaaS”), software-as-a-service (“SaaS”), and/or desktop-as-a-service (“DaaS”). As a non-exclusive example, IaaS may include, e.g., one or more virtual server instantiations that may start, stop, access, and/or configure virtual servers and/or storage centers (e.g., providing one or more processors, storage space, and/or network resources on-demand, e.g., EMC and Rackspace). PaaS may include, e.g., one or more software and/or development tools hosted on an infrastructure (e.g., a computing platform and/or a solution stack from which the client can create software interfaces and applications, e.g., Microsoft Azure). SaaS may include, e.g., software hosted by a service provider and accessible over a network (e.g., the software for the application and/or the data associated with that software application may be kept on the network, e.g., Google Apps, SalesForce). DaaS may include, e.g., providing desktop, applications, data, and/or services for the user over a network (e.g., providing a multi-application framework, the applications in the framework, the data associated with the applications, and/or services related to the applications and/or the data over the network, e.g., Citrix). The foregoing is intended to be exemplary of the types of systems and/or methods referred to in this application as “cloud” or “cloud computing” and should not be considered complete or exhaustive.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

To the extent that formal outline headings are present in this application, it is to be understood that the outline headings are for presentation purposes, and that different types of subject matter may be discussed throughout the application (e.g., device(s)/structure(s) may be described under process(es)/operations heading(s) and/or process(es)/operations may be discussed under structure(s)/process(es) headings; and/or descriptions of single topics may span two or more topic headings). Hence, any use of formal outline headings in this application is for presentation purposes, and is not intended to be in any way limiting.

Throughout this application, examples and lists are given, with parentheses, the abbreviation “e.g.,” or both. Unless explicitly otherwise stated, these examples and lists are merely exemplary and are non-exhaustive. In most cases, it would be prohibitive to list every example and every combination. Thus, smaller, illustrative lists and examples are used, with focus on imparting understanding of the claim terms rather than limiting the scope of such terms.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Although one or more users maybe shown and/or described herein, e.g., in FIG. 1, and other places, as a single illustrated figure, those skilled in the art will appreciate that one or more users may be representative of one or more human users, robotic users (e.g., computational entity), and/or substantially any combination thereof (e.g., a user may be assisted by one or more robotic agents) unless context dictates otherwise. Those skilled in the art will appreciate that, in general, the same may be said of “sender” and/or other entity-oriented terms as such terms are used herein unless context dictates otherwise.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

### High-Level System Architecture

FIG. 1, showing how FIGS. 1-A-1-F are assembled to form a complete view of an entire system, of which at least a portion will be described in more detail. An overview of the entire system of FIG. 1 is now described herein.

### Impact Sensor System 12

Referring now to FIG. 1-A, one or more machines/articles/compositions/processes related to traumatic brain injuries (e.g. traumatic brain injuries (TBI), mild traumatic brain injuries (mTBI), concussions, etc.) are depicted as including impact sensor system 12 to detect, sense, measure, or otherwise determine forces related to impact due to collision of a sports player with one or more other sports players, ground, equipment, game balls or other game devices, objects, etc. in which force is imparted to a specified location of a sports player (e.g. on or near player's head). Although not limiting in nature, a particular sort of player impact that can be of concern is that which may have potential for injury to a player's brain such as a traumatic brain injury. Determination of forces imparted to a player's head can be done through dedection of positive or negative linear or angular velocity, acceleration, jerk, etc. of a player in general, and a player's head (e.g. player's head 12e) in particular. Although this detection may not be a final indicator that a traumatic brain injury has occurred, it can be used along with other factors to flag to a certain degree of accuracy the possibility that a brain injury has occurred with a player.

Such detection of positive or negative linear or angular velocity, acceleration (e.g. six-axis accelerometers), jerk, etc. of a player's head can include impact sensor systems such as having head sensors integrated into sports helmets (e.g. football helmet 12f with face shield 12g for football player 12m, such as for example football helmets with the Riddell Insite Response System (only requires football helmet, does not use or require a face shield), baseball helmet 12k for baseball player 12l, BrainSentry peel-n-stick sensor system that affixes to outer surfaces of helmets such as football helmets and includes a display to alert of status. Other helmets can have impact sensors as well such as hard or soft helmets for other sports such as lacrosse, hockey, bicycling, wrestling, soccer, etc.). Other implementations of impact sensor system 12 can include impact sensors integrated with banded devices (e.g. banded display 12h or wristband 12i), head-bands (e.g. head-band 12j), or glasses (e.g. sports glasses 12n), or skullcaps or beanies such as for soccer (e.g. skullcap or beanie 12d, such as skullcap or beanie Checklight MC-10 by Rebok, which energizes one or more indicator lights on the beanie if an impact threshold has been exceeded). Impact sensors can also be integrated into patch (generally affixed to skin) or button (generally small sensor package) systems for helmeted or non-helmeted sports such as basketball, bicycling, soccer, baseball, etc. (e.g. button or patch sensor 12a worn behind ear 12b, such as X-patch system by X2 Biosystems or conventional button sensors). Other impact sensors can be located in mouth guards such as having a 6-axis accelerometer inside of X-Guard mouth guard by X2 Bioystems. Other impact sensor systems can use RFID-based (radio frequency identification based) sensors with RFID emitters (e.g. RFID emitter 12c) affixed to the player and RFID sensors positioned on the playing field to obtain player linear or angular position, velocity, acceleration, jerk data (e.g. conventional RFID technology provided by Zebra Technologies, Inc.).

Various signalling devices such as lights, displays, or audio emitters can be integrated with the impact sensor system 12 such as into or on helmets, head-bands, wrist-bands, to apprise both sports players and others (such as coach, referee, or trainer) of sports player impact status, such as alerting when impact has been over various predetermined thresholds. The impact sensor system 12 also includes communication and configuration capabilities as further described below. These communication and configuration capabilities allow for transmission of impact data detected by the impact sensor system 12 to other systems described herein and to also receive status and configuration information from these other systems. Through this inter-communication and configuration between various other systems and the impact sensor system 12, not only sports player impact data is detected as with conventional approaches but in addition provision can be made for player testing, status, and access management to be integrated with player impact sensing for updating status and systems configurations therebetween.

### Player Testing System 14

Referring now to FIG. 1, e.g. FIGS. 1-B and 1-E, one or more machines/articles/compositions/processes related to traumatic brain injuries (e.g. TBI, mTBI, concussion, etc.) are depicted as including player testing system 14 to interrogate, analyze, monitor, or otherwise assess, etc. at least to an initial degree cognitive, neurological, (otherwise known as neurocognitive), or other brain-related performance levels of a sports player through neurocognitive testing interaction with the sports player or through physiological monitoring of the sports player. Such brain-related performance assessment can be in particular associated with possible occurrence of concussion or other traumatic brain injury of the sports player due to impact imparted to the sports player. Estimates have included somewhere between 2 to 4 million sports related concussions occurring in the United States per year with an estimated 65% to 85% of these concussions being left unreported at least in the initial few days after a concussion has occurred. The gravity of these estimates is better understood when viewed in the context of other estimates having to do with a condition that affects primarily teens since their brains are still developing a great deal relative to adults in general. Second impact syndrome occurs when a second concussion is experienced within approximately 7 days after a first concussion has occurred. Estimates include an approximate 50% possibility of a second concussion under a second impact syndrome scenario causing death and another approximate 50% possibility of a second impact under a second impact syndrome scenario causing severe brain injury. Integration of the player testing system 14 with the other systems discussed herein seeks to in part address high levels of unreported, undiagnosed cases of sports-related traumatic brain injuries. As described further below, the player testing system 14 is integrated with the impact sensing system 12 and other systems described herein so that coordination between the systems is handled in an automated or semi-automated way such that onfield administration of brain-related performance testing and other responsive measures can be less burdensome than otherwise available. The player testing system 14 can include the following devices to implement testing systems and processes described below through integrated image displays, audio emitters, cameras, audio microphones, tactical input (e.g. touch pad, gesture recognition, etc.): medical instrumentation 14a (e.g. image, infrared, fMRI, CAT, or other scanning devices, etc.) wrist or other banded devices 14b, helmet (e.g. football) 14c with shield/visor 14d, mobile device 14e, computer monitor 14f, wrist display 14g, smart phone 14h, beanie 14i, sports glasses 14j, band (e.g. headband) 14k, behind ear 14l device 14m, sports cap 14n, and implant 14o.

Aspects of neurocognitive testing of a sports player by the player testing system 14 can include numerous types of automated or semi-automated testing such as through assessment of sports player eye-tracking, pupil dilation, pupil alignment, pupil synching, etc. as determined through image recognition or other tracking devices. Examples of conventional eye tracking testing include mild traumatic brain injury (mTBI) testing are discussed at https://www.braintrauma.org/research-at-btf/concussion-diagnostics/, http://www.brainline.org/content/multimedia.php?id=6250, and http://www.forbes.com/sites/robertglatter/2014/12/17/%20new-eye-tracking-technology-promising-as-biomarker-for-brain-injury-and-function/ including recent research indicating that shearing of connections in brain frontal area can cause attention and memory deficits in individuals that have suffered a mTBI. Deficits in attention have been correlated with abnormalities in smooth pursuit eye-movement in individuals with damage to these frontal connections. Since smooth pursuit eye-movement is the ability to track an object that is following a consistent and predictable path, tracking an individual's eye movements can be used to assess whether someone has attention deficits from a head injury shearing frontal brain connections. These conventional eye tracking assessments, which are dynamic tests of attention, such as staying focused on a moving object displayed on a screen, can take a little time as 30 seconds (hundreds of data points a second) versus 20 or 30 minutes for reaction tests that have static interaction, such as “hit the button when you see a yellow triangle” (having a few data points per second at best). The conventional eye tracking tests also do not depend on the motivation level of the participant so test-retest reliability is high since either the participant is tracking or is not tracking compared with other response tests that depend upon more overt participation. Other neurocognitive tests are still useful though to give other perspectives on a sports player's status so eye tracking should not be viewed as the only test available that is any good.

Another type of conventional eye-tracking technology that can be incorporated into the player testing system 14 is associated with Oculogica company, which has studied versions of its technology involving participants watching music videos or television content for about four minutes while the ratio of horizontal to vertical eye movements is measured. In neurologically normal participants, ratios are nearly to 1:1, with horizontal movements essentially equaling vertical movements. With nerve damage or brain swelling pressing nerves, abnormal eye movement ratios can reflect the affected nerve such as occurring with traumatic brain injury (TBI) or mild traumatic brain injury (mTBI also known as concussion). This approach by Oculogica can provide potential for classifying and quantitating extent of brain injury.

Conventional eye tracking glasses technology that can be incorporated into the player testing system 14 can be such as from exemplary companies as Applied Science Laboratories that offers the Mobile Eye-XP Eye Tracking Glasses at http://www.asleyetracking.com/Site/Products/MobileEyeXGGlasses/tabid/70/Default.asp x. Eye tracking can be implemented by the Player Testing System 14 through use of helmet shields (e.g. helmet shield 14d of football helmet 14c) or through sports glasses (e.g. sports glasses 14j) by image projection of an object for the sports player to track with their eyes such as on a portion of the inner surface of a helmet shield or sports glasses lens and also including a miniature camera incorporated in a sports helmet or sports glasses to track eye movement of the sports player. Other implementations for image display and camera capture of eye-movement for eye tracking can use portable devices (e.g. portable devices 14e or 14h), computer monitors (e.g. monitor 14f), or sports bands (e.g. sports band 14k for head, arm, etc. optionally having an e-paper display).

Other sports player testing by the player testing system 14 can include audio recognition of player verbal response feedback, tactile player feedback (e.g. joystick controlled feedback from player), etc. Further non-limiting examples of player testing aspects can include automated or semi-automated implementation of neurocognitive or other brain-related performance testing such as for exemplary purposes, similarly found in one or more portions from conventional testing protocols including, but not limited to, Sway Balance™ iOS mobile software by Sway Medical, LLC, Standardized Assessment of Concussion (SAC), Standardized Concussion Assessment Tool (SCAT2), King-Devick testing system, Balance Error Scoring System (BESS), imPACT™ (Immediate Post-Concussion Assessment and Cognitive Testing) system, Reaction Time including mechanical based testing, Dynamic Visual Acuity Testing, etc. Portions of other conventional neurocognitive protocols that can be used by the player testing system 14 can be in the form of conventional pencil-and-paper test (e.g. similar to SAC or SCAT2) that has been adapted for computer-automated input or tests that have already been computerized such as imPACT testing, as described at https://www.impacttest.com/products/?The-ImPACT-Test-2 as including demographic information, concussion history, learning disabilities, current concussion symptoms, and neurocognitive testing. The imPACT testing further includes neurocognitive testing having word discrimination (attention and verbal recognition memory), design memory (attention and visual recognition memory), “X's and O's” (visual working memory and processing speed), symbol matching (visual processing speed, learning and memory), color match (choice reaction time, impulse control and response inhibition), three letter memory (working memory and visual-motor response speed). Verbal memory, visual memory, processing speed, reaction time and symptom scores are used to determine when a concussion has occurred.

The player testing system 14 can use other forms of neurocognitive testing similar to such conventional neurocogtive testing systems as the automated neuropsychological assessment metrics (ANAM) by Vista Life Science, which according thereto “provides randomized stimuli on tasks that are well-established cognitive measures, and records accuracy and timing of response with millisecond sensitivity and has a special timing mechanism to ensure test/re-test reliability.” as stated at http://www.vistalifesciences.com/index.php/anam-intro.html.

The player testing system 14 can use other forms of neurocognitive testing similar to such conventional neurocogtive testing systems as that of HeadMinder by HeadMinder, Inc., which according thereto “consists of a set of computerized subtests that require simple patient responses on a standard keyboard and measure aspects of cognition typically associated with a brain dysfunction, such as reaction time, concentration and working memory, information processing speed and accuracy, and short-term and long-term memory. Tests may also include questionnaires tailored for each presenting problem . . . . HeadMinder scientists have implemented the only existing commercial system that uses advanced statistical models for measuring and monitoring change in cognitive functions. Each individual's initial test results are used as a baseline for comparison to future tests. The baseline allows the system to create a unique longitudinal profile: the individual is compared to himself or herself over time, thereby increasing the accuracy of the test. (Most traditional assessment measures compare individuals to a group average.) . . . Specialized statistics control for practice effects and reduce other sources of error. Our tests employ multiple alternate forms, and our server keeps track of which forms have already been administered.”

The player testing system 14 can use other forms of neurocognitive testing similar to such conventional neurocogtive testing systems as that of computerized cognitive testing by CogState Research http://cogstate.com/academic-2/measurement-of-cognition/#.VNrxMi62Jm4, which includes Visual Motor Function (Chase Test), Executive Function/Spatial Problem Solving (Groton Maze Learning Test and Set-Shifting Task), Psychomotor Function/Speed of Processing (Detection Task), Visual Attention/Vigilance (Identification Task), Visual Learning & Memory (One Card Learning Task, Continuos Paired Associated Learning Task, Groton Maze Learning Test—Delayed Recall), Verbal Learning & Memory (International Shopping Lisk Task and International Shopping List Task: Delayed Recall), Attention/Working Memory (One Back Task and Two Back Task) Social Cognition (Social-Emotional Cognition Task).

These and other sports player testing protocols can be implemented through automated or semi-automated devices as part of sports player testing by the player testing system 14 to arrive at initial brain-related performance assessments. Hybrid combinations of these or other assessments can also be used as implementations of existing and other sports player testing protocols. In addition, electronic image recognition of electronic image data or electronic audio recognition of electronic audio data of sports player behavior, expression, or other sorts of output by the sports player can be performed. Electronic image or audio data related to sports player behavior, expression, or other output can be captured for electronic image recognition or electronic audio recognition in various locations. Examples of such locations for such sports player behavior can be either on or off the field of play (e.g. field, court, rink, mat, etc.) during play of a game, during game intermission or other break in play of game, during down-time of a sports player away from the game area (e.g. away from sports field, court, rink, etc.) on the sidelines or elsewhere off of the field, or during play of game by other sports players, during implementation of testing protocols with a sports player or at other times can also be used to complement or otherwise furnish testing input to assess neurocognitive or other brain-related performance status for the sports player.

The player testing system 14 can also use sports player implanted devices or other physiological blood testing devices to monitor indicators of TBI, mTBI, concussion, etc. For instance, use of serum biomarkers such as S-100B, GFAP, neuron specific enolase (NSE), etc. could potentially be used in part to evaluate sports players after a potentially brain-injurious impact. For instance, elevated levels of S-100B may have some use in predicting severity of a brain injury later after injury as discussed at http://www.forbes.com/sites/robertglatter/2013/11/02/seattle-based-company-x2-biosystems-poised-to-change-approach-to-evaluation-of-traumatic-brain-injury-and-concussions/3/. Other blood testing by the player testing system 14 could include testing for elevated levels in the blood of the brain-enriched protein calpain-cleaved αII-spectrin N-terminal fragment, known as SNTF, to predict severity of symptoms due to a brain-injurious impact to a sports player as discussed at http://www.uphs.upenn.edu/news/News_Releases/2014/11/concussion/.

The player testing system 14 also includes communication and configuration capabilities as further described below. These communication and configuration capabilities allow for transmission of data related to player testing as determined by the player testing system 14 to other systems described herein and to also receive status and configuration information from these other systems. Through this inter-communication and configuration between various other systems and the player testing system 14, not only player testing data is determined as with conventional approaches but in addition provision can be made for player impact sensing, status, and access management to be integrated with player testing for updating status and systems configurations therebetween.

### Player Status System 16

Referring now to FIG. 1-D, one or more machines/articles/compositions/processes related to traumatic brain injuries (e.g. traumatic brain injuries (TBI), mild traumatic brain injuries (mTBI), concussions, etc.) are depicted as including impact sensor system 12 to report, broadcast, signal, alert, update, inform, or otherwise notify users and those otherwise associated regarding status of one or more players being monitored, etc. by impact sensor system 12, player testing system 14, field access system 18, or player location system 20. Past, present, or predicted metrics inputted or derived from these various systems and other ad hoc inputs or systems sent to the player status system 16 are then presented to individuals and groups accordingly, such as including output of status for one or more players, fans, officials, reporters, spectators, parents, referees, etc. Outputs and participants of the player status system 16 can include, but are not limited to scoreboard output 16a, baseball participant 16b, football participant 16c, coaching staff participant 16d, medical trainer participant 16e, referee participant 16f, monitor output 16g, instrument output 16h, helmet output 16i, wrist output 16j, watch output 16k, band output 16l, cap output 16m, handheld output 16n, auditory output 16o, button-sized output 16p, hat output 16q, glasses output 16r, signal output 16s, speaker output 16t, emitter output 16u, and visor output 16v.

Status of the player status system 16 can include player position, impact experienced by player, player TBI testing status, data-based information, current and historical player field-position information, current and historical impact information, current and historical TBI testing information, etc. The player status system 16 can include query capabilities to retrieve additional position, impact, testing and other information. The player status system 16 can also include a user-interface that can be customized according to user status such as user being player, coach, trainer, medic, parent, referee, scoreboard, medic, hospital, etc. User interfaces can be integrated into hand-held, laptop, mobile, stand-based, or table-based devices. Communication involved with the player status system 16 can include such as when system transmits/receives information/data to/from impact sensor system, system transmits/receives information/data to/from player testing system, and system transmits/receives information/data to/from field access system.

### Field Access System 18

Referring now to FIG. 1-C, one or more machines/articles/compositions/processes related to traumatic brain injuries (e.g. traumatic brain injuries (TBI), mild traumatic brain injuries (mTBI), concussions, etc.) are depicted as including field access system 18 to receive player information regarding status of one or more players being monitored, etc. by impact sensor system 12, player testing system 14, player status system 16, or player location system 20. Past, present, or predicted metrics inputted or derived from these various systems and other ad hoc inputs or systems sent to the field access system 18 are then used to determine access parameters to use to control access of one or more players, officials, other personnel, or persons via the field access system 18. Controls are used to either physically bar or informationally bar recipients of the controls from entering into restricted areas such as an area of a playing field or an entire playing field or an entire stadium complex, etc. Controls using information to bar access can output information related to a player access control either visually, auditory means, tactical means, or other means, which can be integrated into clothing, gear, etc. either affixed to player(s) or separate from player(s). Control aspects of the field access system 18 can include, but are not limited to computer control 18a, stadium control 18b, signal control 18c, speaker control 18d, emitter control 18e, gate control 18f, auditory control 18g, button control 18h, hat control 18i, helmet control 18j, visor control 18k, wrist control 18l, watch control 18m, band control 18n, cap control 18o, or glasses control 18p.

The field access system 18 can include controlling player access to: football, soccer, lacrosse, baseball, fields, etc., basketball, squash, racquetball courts, etc., wrestling mats, hockey rinks, etc. The field access system can include physical, electromagnetic, audio, or visual based player barriers in which physical barriers can include gated access to field, electromagnetic can include irritant-based output, audio can include directed interfering sound to player, visual can include blocked, altered, irritated player vision such as with helmet shield, glasses, goggles can include light-based or audio-based, field-mounted signaling to coach, referee, trainer, players, fans, etc., wearable signaling such as by arm, wrist, head, trunk, etc. and mounted signaling. As show the field access system 18 transmits/receives information/data to/from impact sensor system, transmits/receives information/data to/from player status system, or transmits/receives information/data to/from field access system.

Player Location System 20

Referring now to FIG. 1-F, one or more machines/articles/compositions/processes related to traumatic brain injuries (e.g. traumatic brain injuries (TBI), mild traumatic brain injuries (mTBI), concussions, etc.) are depicted as including player location system 20 to receive player information regarding status of one or more players being monitored, etc. by impact sensor system 12, player testing system 14, player status system 16, or player access system 18. Past, present or predicted metrics inputted or derived from these various systems and other ad hoc inputs or systems sent to the player location system 20 are then used to assess various requirements for player location information determined by the player location system 20. Player location is sent to these systems for their use and for use by players, officials, other personnel, or other persons. Location aspects of the player location system 20 can include, but are not limited to computer locator 20a, stadium locator 20b, watch locator 20c, band locator 20d, wrist locator 20e, helmet locator 20f, visor locator 20g, RFID locator 20h, emitter locator 20i, in ground locator 20j with buried perimeter wires 20k, 20l, and 20m.

In operation, the player location system 20 includes such functions as determining current and historical player field-location data. The player location system 20 can include use of RF emitter with spaced wire-pair. The wire pair can be buried in ground. The player location system 20 can determine player location through RFID based systems. The player location system can include technology such as based on Zebra Technologies RFID system player emitters and field sensors. The player location system 20 can include player location determination thru player image recognition, player uniform recognition, or player-specific spectrum based recognition. The player location system 20 can transmit/receive information/data to/from field access system, etc.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

To the extent that formal outline headings are present in this application, it is to be understood that the outline headings are for presentation purposes, and that different types of subject matter may be discussed throughout the application (e.g., device(s)/structure(s) may be described under process(es)/operations heading(s) and/or process(es)/operations may be discussed under structure(s)/process(es) headings; and/or descriptions of single topics may span two or more topic headings). Hence, any use of formal outline headings in this application is for presentation purposes, and is not intended to be in any way limiting.

Throughout this application, examples and lists are given, with parentheses, the abbreviation “e.g.,” or both. Unless explicitly otherwise stated, these examples and lists are merely exemplary and are non-exhaustive. In most cases, it would be prohibitive to list every example and every combination. Thus, smaller, illustrative lists and examples are used, with focus on imparting understanding of the claim terms rather than limiting the scope of such terms.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Although one or more users maybe shown and/or described herein, e.g., in FIG. 1, and other places, as a single illustrated figure, those skilled in the art will appreciate that one or more users may be representative of one or more human users, robotic users (e.g., computational entity), and/or substantially any combination thereof (e.g., a user may be assisted by one or more robotic agents) unless context dictates otherwise. Those skilled in the art will appreciate that, in general, the same may be said of “sender” and/or other entity-oriented terms as such terms are used herein unless context dictates otherwise.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

This application may make reference to one or more trademarks, e.g., a word, letter, symbol, or device adopted by one manufacturer or merchant and used to identify and/or distinguish his or her product from those of others. Trademark names used herein are set forth in such language that makes clear their identity, that distinguishes them from common descriptive nouns, that have fixed and definite meanings, or, in many if not all cases, are accompanied by other specific identification using terms not covered by trademark. In addition, trademark names used herein have meanings that are well-known and defined in the literature, or do not refer to products or compounds for which knowledge of one or more trade secrets is required in order to divine their meaning. All trademarks referenced in this application are the property of their respective owners, and the appearance of one or more trademarks in this application does not diminish or otherwise adversely affect the validity of the one or more trademarks. All trademarks, registered or unregistered, that appear in this application are assumed to include a proper trademark symbol, e.g., the circle R or bracketed capitalization (e.g., [trademark name]), even when such trademark symbol does not explicitly appear next to the trademark. To the extent a trademark is used in a descriptive manner to refer to a product or process, that trademark should be interpreted to represent the corresponding product or process as of the date of the filing of this patent application.

Throughout this application, the terms “in an embodiment,” ‘in one embodiment,” “in some embodiments,” “in several embodiments,” “in at least one embodiment,” “in various embodiments,” and the like, may be used. Each of these terms, and all such similar terms should be construed as “in at least one embodiment, and possibly but not necessarily all embodiments,” unless explicitly stated otherwise. Specifically, unless explicitly stated otherwise, the intent of phrases like these is to provide non-exclusive and non-limiting examples of implementations of the invention. The mere statement that one, some, or may embodiments include one or more things or have one or more features, does not imply that all embodiments include one or more things or have one or more features, but also does not imply that such embodiments must exist. It is a mere indicator of an example and should not be interpreted otherwise, unless explicitly stated as such.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

What is claimed is:

1. A method comprising:
a method substantially as shown and described in the detailed description and/or drawings and/or elsewhere herein.

2. A device comprising:
a device substantially as shown and described in the detailed description and/or drawings and/or elsewhere herein.
